(19) United States
(12) Patent Application Publication
(10) Pub. No.: US 2024/0075098 A1
(43) Pub. Date: Mar. 7, 2024

(54) COCHLEAR OUTER HAIR CELL REGENERATED BY ECTOPIC JOINT OVEREXPRESSION OF ATOH1 AND IKZF2 AND APPLICATION THEREOF

(71) Applicant: CENTER FOR EXCELLENCE IN BRAIN SCIENCE AND INTELLIGENCE TECHNOLOGY, CHINESE ACADEMY OF SCIENCES, Shanghai (CN)

(72) Inventors: Zhiyong LIU, Shanghai (CN); Suhong SUN, Shanghai (CN); Shuting LI, Shanghai (CN); Zhengnan LUO, Shanghai (CN); Minhui REN, Shanghai (CN); Shunji HE, Shanghai (CN); Guangqin WANG, Shanghai (CN)

(21) Appl. No.: 18/260,527

(22) PCT Filed: Feb. 22, 2021
(86) PCT No.: PCT/CN2021/077285

§ 371 Date: Jul. 6, 2023

(30) Foreign Application Priority Data
Jan. 7, 2021 (CN) 202110018325.2

(51) Int. Cl. A61K 38/17; A61P 27/16; C12N 15/63

(52) U.S. Cl. CPC A61K 38/1709; A61P 27/16; C12N 15/63

(57) ABSTRACT

The present invention provides a cochlear outer hair cell (OHC) regenerated by ectopic joint overexpression of Atoh1 and Ikzf2 and an application thereof. Specifically, the present invention provides an active ingredient combination capable of being used for regenerating a cochlear OHC, and a use thereof. The active ingredient combination comprises an Ikzf2 protein and an Atoh1 protein; and after the active ingredient combination is administered to a subject suffering from hearing impairment related to cochlear OHC degeneration and/or injury, hearing impairment can be alleviated by regenerating an OHC-like cell. A new method is provided for clinical treatment of hearing impairment.

### TECHNICAL FIELD

The present invention relates to the fields of biological preparations and biotechnology, in particular to the cochlea outer hair cell regenerated by combined overexpression of Atoh1 and Ikzf2 and applications thereof.

### BACKGROUND

The outer hair cells (OHCs) of the mammalian cochlea are important for hearing, but OHCs are very sensitive to various ototoxic drugs, noise and aging. Degeneration or damage of OHC leads to severe hearing disorders. In previous in vivo studies, there have been precedents for treating hearing impairment through OHC regeneration, however, its efficiency is low, and the regenerated new OHCs are usually non-functional.

Therefore, there is an urgent need to develop a method for regenerating OHC in the presence of OHC degeneration or damage in the body to treat hearing impairment in this field.

### SUMMARY OF THE INVENTION

The purpose of the present invention is to provide a genetic treatment method and therapeutic drug that can effectively treat hearing impairment.

In the first aspect of the present invention, it provides a use of a combination of active ingredients, for preparing a preparation or drug, which is used for (i) regenerating cochlea outer hair cells, and/or (ii) treating or preventing hearing impairment;
wherein the combination of active ingredients comprises:
(a) Ikzf2 protein, a coding sequence thereof, or a promoting agent thereof, or a combination thereof; and
(b) Atoh1 protein, a coding sequence thereof, or a promoting agent thereof, or a combination thereof.

In another preferred embodiment, the IKZF2 protein has an amino acid sequence as shown in SEQ ID NO. 1.

In another preferred embodiment, the Atoh1 protein has an amino acid sequence as shown in SEQ ID NO.: 2.

In another preferred embodiment, the hearing impairment is related to the degeneration and/or damage of cochlea outer hair cells (OHCs).

In another preferred embodiment, the cochlea outer hair cells are cochlea outer hair cells of human or non-human mammalian.

In the second aspect of the present invention, it provides a combination of active ingredients that can be used to regenerate cochlea outer hair cells, comprising:
(a) Ikzf2 protein, a coding sequence thereof, or a promoting agent thereof, or a combination thereof; and
(b) Atoh1 protein, a coding sequence thereof, or a promoting agent thereof, or a combination thereof.

In another preferred embodiment, the IKZF2 protein has an amino acid sequence as shown in SEQ ID NO.: 1.

In another preferred embodiment, the Atoh1 protein has an amino acid sequence as shown in SEQ ID NO.: 2.

In the third aspect of the present invention, it provides an expression vector comprising:
(Z1) a first expression vector containing a first expression cassette used to express Ikzf2 protein;
(Z1) a second expression vector containing a second expression cassette used to express Atoh1 protein;
wherein the first expression vector and the second expression vector are the same vector or different vectors.

In another preferred embodiment, the expression vector includes a viral vector.

In another preferred embodiment, the expression vector is selected from the group consisting of a plasmid and a viral vector.

In another preferred embodiment, the expression vector is selected from the group consisting of a lentiviral vector, an adenovirus vector, an adeno-associated viral vector (AAV), and a combination thereof.

In another preferred embodiment, the expression vector is an adeno-associated viral vector (AAV).

In another preferred embodiment, the AAV vector is AAV7m8, AAV-anc80, or AAV-ie.

In another preferred embodiment, the expression vector is used to express Ikzf2 protein and/or Atoh1 protein.

In another preferred embodiment, the expression vector contains a nucleotide sequence encoding Ikzf2 protein and/or Atoh1 protein.

In another preferred embodiment, the nucleotide sequence encoding Ikzf2 protein is as shown in SEQ ID NO.: 3.

In another preferred embodiment, the nucleotide sequence encoding Atoh1 protein is as shown in SEQ ID NO.: 4.

In another preferred embodiment, the first expression cassette has a structure from the 5′-terminus to 3′-terminus as shown in Formula I:

Z0-Z1-Z2 (I)
wherein,
each “−” independently is a chemical bond or a nucleotide linker sequence;
Z0 is none, or a 5′UTR sequence;
Z1 is a nucleotide sequence encoding Ikzf2 protein; and
Z2 is none, or a 3′UTR sequence.

In another preferred embodiment, the second expression cassette has a structure from the 5′-terminus to 3′-terminus as shown in Formula II:

Z0′-Z1′-Z2′ (II)
wherein,
each “−” independently is a chemical bond or a nucleotide linker sequence;
Z0′ is none, or a 5′UTR sequence;
Z1′ is a nucleotide sequence encoding Atoh1 protein; and
Z2′ is none, or a 3′UTR sequence.

In another preferred embodiment, each nucleotide linker sequence is 1-30 nt, preferably 1-15 nt, and more preferably 3-6 nt in length.

In another preferred embodiment, the nucleotide linker sequence is derived from a nucleotide linker sequence formed by restriction endonuclease digestion.

In the fourth aspect of the present invention, it provides a host cell containing the expression vector of the third aspect of the present invention.

In another preferred embodiment, the host cells are mammalian cells, wherein mammals include human and non-human mammals.

In another preferred embodiment, the host cells are selected from the group consisting of cochlea supporting cells (SCs), including pillar cells (PCs), Deiters cells (DCs) and a combination thereof; preferably pillar cells (PCs).

In the fifth aspect of the present invention, it provides a pharmaceutical preparation that contains (a) the combination of active ingredients of the second aspect of the present invention, or the vector of the third aspect of the present invention, or the cell of the fourth aspect of the present invention, and (b) a pharmaceutically acceptable carrier or excipient.

In another preferred embodiment, the dosage form of the pharmaceutical preparation is selected from the group consisting of a freeze-dried preparation, liquid preparation, and a combination thereof.

In another preferred embodiment, the vector is selected from the group consisting of a lentiviral vector, an adenoviral vector, an adeno-associated viral vector, and a combination thereof; preferably, the vector is an AAV vector; more preferably AAV7m8, AAV-anc80, or AAV-ie.

In another preferred embodiment, the content of the vector in the pharmaceutical preparation is 1×10¹²-1×10¹⁴ viruses/ml, preferably 1×10¹³ viruses/ml.

In another preferred embodiment, the therapeutic effective amount of the pharmaceutical preparation is 1×10⁹-1×10¹¹ viruses, preferably 1×10¹⁰ viruses.

In another preferred embodiment, the pharmaceutical preparation is used for treating or preventing hearing impairment, preferably treating the degeneration and/or damage of cochlea outer hair cells.

In the sixth aspect of the present invention, it provides a use of the combination of active ingredients of the second aspect of the present invention, the expression vector of the third aspect of the present invention, the host cell of the fourth aspect of the present invention, or the pharmaceutical preparation of the fifth aspect of the present invention in the treatment or prevention of hearing impairment.

In another preferred embodiment, the hearing impairment is related to the degeneration and/or damage of cochlea outer hair cells (OHCs).

In the seventh aspect of the present invention, it provides a method of treating or preventing hearing impairment, wherein the method comprises administrating the pharmaceutical formulation of the fifth aspect of the present invention to a subject in need thereof.

In another preferred embodiment, the hearing impairment is related to the degeneration and/or damage of cochlea outer hair cells.

In another preferred embodiment, the method is a method to reduce the degeneration and/or damage of cochlea outer hair cells in a patient with or at risk of developing hearing impairment.

In another preferred embodiment, the subject in need thereof includes human and non-human mammals.

In another preferred embodiment, the subject in need thereof suffers from hearing impairment related to the degeneration and/or damage of cochlea outer hair cells.

In another preferred embodiment, the method comprises directly administrating the vector of the present invention into the ear of the subject in need thereof.

In another preferred embodiment, the method comprises directly administrating the host cells of the present invention into the cochlea of the subject in need thereof.

In another preferred embodiment, the method can regenerate the cochlea outer hair cells of the subject in need thereof with hearing impairment related to the degeneration and/or damage of cochlea outer hair cells.

In another preferred embodiment, the method enables the hearing function in the processed ears to be restored or maintained.

In the eighth aspect of the present invention, it provides a method of regenerating cochlea outer hair cells, comprising transducing the combination of active ingredients of the second aspect of the present invention, or the expression vector of the third aspect of the present invention into cochlea supporting cells.

In another preferred embodiment, the cochlea supporting cells are pillar cells (PCs), including Deiters cells (DCs) and a combination thereof.

In the ninth aspect of the present invention, it provides a use of an active ingredient for preparing a preparation or drug, which is used for (i) promoting the regeneration of cochlea outer hair cells, and/or (ii) auxiliary treatment or prevention of hearing impairment;
wherein the active ingredient includes: Ikzf2 protein, a coding sequence thereof, or a promoting agent thereof, or a combination thereof.

In another preferred embodiment, the IKZF2 protein has an amino acid sequence as shown in SEQ ID NO. 1.

In another preferred embodiment, the hearing impairment is related to the degeneration and/or damage of cochlea outer hair cells (OHCs).

In another preferred embodiment, the cochlea outer hair cells are cochlea outer hair cells of human or non-human mammalian.

It should be understood that within the scope of the present invention, each technical features of the present invention described above and in the following (such as examples) may be combined with each other to form a new or preferred technical solution, which is not listed here due to space limitations.

### DESCRIPTION OF THE DRAWINGS

FIG. 1: After ectopic overexpression of Ikzf2 in the inner hair cells, the inner hair cells began to express Prestin. (A) Illustration shows how to initiate ectopic expression of Ikzf2 in hair cells. Tdtomato and Ikzf2 (HA tag) are associated with expression. Atoh1-CreER+ is an effective initiation tool for early hair cells. (B-C′″) Prestin, vGlut3 and Tdtomato were stained simultaneously in the control group Atoh1-CreER+(B-B′″) and the experiment group Atoh1-CreER+; Rosa26-CAG-LSL-Ikzf2/+(C-C′″) of P42. Among them, Prestin is only expressed in wild outer hair cells (B-B′″). In (C-C″), the arrow points out the inner hair cells that expressed Tdtomato/vGlut3/Prestin at the same time, and the asterisk shows the inner hair cells that did not express Prestin in the experimental group. (D) Quantitative analysis of Prestin positive hair cells.** p<0.01. (E-F) HA/Tdtomato were co-stained in the control group (E) and the experimental group (F) of P42. Scale Bars: 20 μm.

FIG. 2: Overexpression of Atoh1 and Ikzf2 in both PCs and DCs of adult mice can generate a small amount of OHC like cells. In the four types of mice treated with TMX at P30 and P31, and analyzed at P60, HA, Tdtomato and Prestin were stained at the same time. (A-A′″) In Fgfr3-iCreER+; Ai9/+ (Fgfr3-Ai9) mice, all Tdtomato positive cells are supporting cells (SCs): PCs and DCs. Embedded in (A′) is the supporting cell layer. The arrow marks a Prestin+/Tdtomato− outer hair cell. (B-B′″) There is neither Tdtomato signal nor Prestin+/HA+double positive signal in Fgfr3-iCreER+; CAG-LSL-Atoh1+ (Fgfr3-Atoh1) mice. The graph embedded in (B) shows the supporting cell layer. (C-D′″) In Fgfr3-iCreER+; Rosa26-CAG-LSL-Ikzf2+ (Fgfr3-Ikzf2) mice, (C-C′″) shows the hair cell layer and (D-D′″) shows the supporting cell layer. The arrows indicate the HA+/Tdtomato+, Pestin− cells in both two layers, while outer hair cells of Prestin+ in situ are abnormal. (E-F′″) The hair cell (E-E′″) and supporting cell (F-F′″) layers in Fgfr3-iCreER+; CAG-LSL-Atoh1+; Rosa26-CAG-LSL-Ikzf2/+ (Fgfr3-Atoh1-Ikzf2) mice. According to location and morphology, the arrows in (E-E′″) indicate HA+/Tdtomato+/Prestin+ OHC-like cells transformed from adult DCs, and the arrows in (F-F′″) indicate HA+/Tdtomato+/Prestin+ OHC-like cells transformed from adult PCs. The arrows mark that in situ Prestin+/Tdtomato− outer hair cells fall into the supporting cell layer. Obviously, the signal of Prestin in OHC-like cells is weaker than that of outer hair cells in situ. (G) Quantitative analysis of OHC-like cells in Fgfr3-Atoh1-Ikzf2 mice. Mean±SEM (n=3) was used to analyze the data. (H) Summarization of the reprogramming results of the three models. Scale Bars: 20 μm.

FIG. 3: Specific damage to outer hair cells by genetic and pharmacological methods. (A-B′) Prestin-P2A-DTR/+ was treated diphtheria toxin (DT) at P36 (A-A′, the control group not present) or (B-B′, the experiment group present), and analyzed at P42. Samples were stained with both Prestin and vGlut3, and the white box regions in (A) and (B) were shown in (A′) and (B′). (Arrows in B′) shows that after 6 days of administration, most outer hair cells died rapidly, leaving a large number of green residual fragments and only a small number of remaining outer hair cells (arrows in B′). (C) Auditory brainstem response of Prestin-P2A-DTR/+ mice, control group (blue line, without DT) and experimental group (red line, with DT). (D) The ratio of outer hair cells to inner hair cells in the experimental group (red line) and the control group (blue line). Wherein, Scale bars: 200 μm (B), 20 μm (B′).

FIG. 4: After in situ injury to outer hair cells, it can promote the reprogramming efficiency of overexpression of Atoh1 and Ikzf2 in adult supporting cells. (A) For different cell models, TMX was administrated at P30 and 31, DT was administrated at P36, and analyzed at P60. (B) Cartoon diagram explains that at the cellular level, Tdtomato can permanently label supporting cells after overexpression of Atoh1 and Ikzf2 in adult PCs and DCs. (C-G′″) In 4 different types of mice: 1) Prestin-DTR/+(C and D), 2) Fgfr3-iCreER+; CAG-LSL-Atoh1+; Prestin-DTR/+(Fgfr3-Atoh1-DTR) (E), 3) Fgfr3-iCreER+; Rosa26-CAG-LSL-Ikzf2/+; Prestin-DTR/+ (Fgfr3-Ikzf2-DTR) (F), and 4) Fgfr3-iCreER+; CAG-LSL-Atoh1+; Rosa26-CAG-LSL-Ikzf2/+; Prestin-DTR/+ (Fgfr3-Atoh1-Ikzf2-DTR) (G-G″″), HA, Tdtomato and Prestin were simultaneously stained. At P60, compared with intact hair cells (C) in Prestin-DTR/+ mice without DT treatment, only a small number of hair cells remained in DT-treated mice (arrows in D). Compared with P42 (FIG. 2), the fragments of dead outer hair cells disappeared. Although no OHC-like cells were generated in the first three mouse models, Tdtomato+/HA+/Prestin+ OHC-like cells were generated in the Fgfr3-Atoh1-Ikzf2-DTR mouse model (arrows in G′″) (H) Quantitative analysis of the number of OHC-like cells in Fgfr3-Atoh1-Ikzf2-DTR mice. (I) Comparison of the number of OHC-like cells in Fgfr3-Atoh1-Ikzf2-DTR and Fgfr3-Atoh1-Ikzf2 mouse models (without destruction of in situ outer hair cells). Wherein, Scale bars: 20 μm.

FIG. 5: Scanning electron microscope (SEM) analysis results of OHC-like cells. (A-A′) In Prestin-DTR/+ mice without DT treatment, the outer hair cells show V or W shaped cilium bundles. (A′) shows an enlarged view of the black box region in (A). (B) In Prestin-DTR/+ mice to which DT had been given at P36, the black asterisk indicates a missing inner hair cell. (C) Irregular cilium bundles were observed in the model of Fgfr3-iCreER+; CAG-LSL-Atoh1+; Rosa26-CAG-LSL-Ikzf2+; Prestin-DTR/+ (Fgfr3-Atoh1-Ikzf2-DTR). They should come from OHC-like cells. The black box (C′) shows an enlarged view in (C). The cilium bundles in the figure embedded in C are rarely observed, and the black asterisk indicates a missing inner hair cell. Wherein, Scale bars: 5 m (A, B, C), 1 μm (C′) and 500 nm (A′).

FIG. 6: OHC-like cells are similar to wild-type outer hair cells of P1. (A) Cartoon images of hand-picked Tdtomato+ cells from three mouse models and single cell sequencing. (B-C′″) Myo7a, Prestin and Tdtomato were stained in the cochlear samples of P60 control (B) and Fgfr3-Atoh1-Ikzf2-DTR (C) mice. The arrows point out the OHC-like cells of Tdtomato+/Myo7a+/Prestin+, and the arrows point out the cells of Tdtomato+/Myo7a−/Prestin−. The asterisk marks the cells of Tdtomato+/Myo7a+/Prestin−(C″-C′″). (D) UMAP analysis of five cell types, in which 42 Tdtomato+ cells marked with light blue dashed lines came from the mouse model of Fgfr3-Atoh1-Ikzf2-DTR of P60, and they were divided into three subgroups. (E) Pearson correlation analysis of 5 different cell types in (D). (F-G) UMAP analysis of cell types (wild-type outer hair cells: supporting cells of E16, P1, P7, P30, and P60, reprogrammed hair cells) in (D-E) except supporting cells that cannot be transformed into hair cells.

### DETAILED DESCRIPTION

After extensive and in-depth research, the present inventor successfully transformed adult cochlear SCs (mainly pillar cells and Deiters cells) into Prestin+ OHC-like cells for the first time by ectopic expression of Atoh1 and Ikzf2, which are two key transcription factors (TF) necessary for OHC development. The transformed OHC-like cells up-regulated hundreds of OHC genes and down-regulated their original SC genes, respectively. Through single cell transcriptome comparison, it was found that OHC-like cells are very similar to wild-type OHCs. Summing up, a novel and effective method for regenerating OHCs in damaged cochlea was established in the present invention, which has clinical potential for regenerating OHCs.

On this basis, the present invention has been completed.

### Term

In order to make this disclosure easier to understand, certain terms are first defined. As used herein each of the following terms shall have the meanings given below unless expressly provided herein. Other definitions are set out throughout the application.

The term “about” may refer to a value or composition within the acceptable error range of a particular value or composition as determined by those skilled in the art, which will depend in part on how the value or composition is measured or determined. For example, as used herein, “about 100” includes all values between 99 and 101 (e.g., 99.1, 99.2, 99.3, 99.4, etc.).

As used herein, the term “contains” or “includes (comprises)” can be open, semi-closed and closed. In other words, the term also includes “substantially consisting of . . . ” or “consisting of . . . ”.

As used herein, the terms “subject”, “subject in need” refer to any mammal or non-mammal. Mammals include, but are not limited to, humans, vertebrates such as rodents, non-human primates, cattle, horses, dogs, cats, pigs, sheep, goats.

As used herein, the terms “hearing impairment”, “hearing disorder” and “hearing injury” refer to hearing dysfunction due to degeneration and/or damage of cochlea outer hair cells.

Cochlea Outer Hair Cells (OHCs)

As used herein, the terms “cochlea outer hair cell”, “OHC” and “outer hair cell” have the same meaning and can be used interchangeably herein.

Sound receptor hair cells (HCs) of a mammal are located in the auditory epithelium and are called Corti organs (OC). Close to HC are several subtypes of supporting cells (SCs), from medial to lateral, pillar cells (PCs) and Deiters cells (DCs), respectively. There are two subtypes of auditory HCs: inner hair cells (IHCs) and outer hair cells (OHCs). OHCs specifically express a unique motor protein Prestin encoded by Slc26a5. Prestin-mediated electrical motion makes OHC a sound amplifier, which is very important for sound detection. Prestin−/− mice suffered severe hearing impairment. Unlike OHCs, IHCs are primary sensory cells innervated by cochlear spiral ganglion neurons (SGNs). IHC specificity is determined by vGlut3 encoded by Slc17a8, which is required for voice information conversion from IHCs to SGNs. The vGlut3−/− mice were completely deaf. IHCs and OHCs share the same Atoh1+ progenitor cells.

Ikzf2 Protein

Ikzf2 (IKAROS Family Zinc Finger 2) is a zinc finger protein, and another common name of it is Helios. For specific information, please refer to:
https://www.ncbi.nlm.nih.gov/gene/22807 (human);
https://www.ncbi.nlm.nih.gov/gene/22779 (mouse).

In the present invention, the amino acid sequences of the Ikzf2 protein are shown SEQ ID NO.: 1 (human) and SEQ ID NO.: 5 (mouse). The nucleotide coding sequences are shown in SEQ ID NO.: 3 (human) and SEQ ID NO.: 7 (mouse).

Ikzf2 is essentially a transcription factor distributed in the nucleus, and its main function is to initiate the expression of specific genes. In the mouse cochlea, Ikzf2 begins to express after birth, and is specifically expressed in the outer hair cells (OHCs) of the cochlea. Other cells of the cochlea do not express or express it in a very low amount. The Ikzf2 mutant mice show abnormal development of cochlea outer hair cells and hearing impairment.

Atoh1 Protein

Atoh1 (atonal bHLH transcription factor 1) is a nuclear transcription factor of the bHLH family, and its another commonly used name is math1. For specific information, please refer to:
https://www.ncbi.nlm.nih.gov/gene/474 (human);
https://www.ncbi.nlm.nih.gov/gene/11921 (mouse).

In the present invention, the amino acid sequences of the Atoh1 protein are shown SEQ ID NO.: 2 (human) and SEQ ID NO.: 6 (mouse). The nucleotide coding sequences are shown in SEQ ID NO.: 4 (human) and SEQ ID NO.: 8 (mouse).

Atoh1 is also essentially a transcription factor distributed in the nucleus, and its main function is to initiate the expression of specific genes. In the mouse cochlea, Atoh1 begins to express in the middle embryonic cochlea, and its main function is to allow the progenitor cells of the cochlear auditory epithelium to develop into hair cells. Atoh1 deficient mice could not generate hair cells.

Adeno-Associated Virus

Adeno-associated virus (AAV), also known as adeno-accompanied virus, belongs to the parvoviridae dependent virus genus. It is a kind of single stranded DNA deficient virus with the simplest structure found at present and needs Helper virus (usually adenovirus) to participate in replication. It encodes cap and rep genes in the two terminal inverted repeat sequences (ITR). ITR has a decisive effect on the replication and packaging of the virus. The cap gene encodes the viral capsid protein, while the rep gene is involved in virus replication and integration. AAV can infect multiple types of cells.

The recombinant adeno-associated virus vector (rAAV) is derived from the non pathogenic wild-type adeno-associated virus, which is regarded as one of the most promising gene transfer vectors, and has been widely used in gene therapy and vaccine research worldwide, due to its good safety, wide host cell range (split and non split cells), low immunogenicity, long duration of expressing foreign genes in vivo and the like. After more than 10 years of research, the biological characteristics of the recombinant adeno-associated virus have been deeply understood, especially in terms of its application effects in various cells, tissues, and in vivo experiments. In medical research, rAAV is used for gene therapy of various diseases (including in vivo and in vitro experiments). As a characteristic gene transfer vector, it is also widely used in gene function research, disease model construction, and gene knockout mouse preparation.

Expression Vector and Host Cells

The present invention provides an expression vector for Ikzf2 protein and Atoh1 protein expression, which contains the encoding sequence of the Ikzf2 protein and Atoh1 protein of the present invention.

Through the provided sequence information, skilled technicians can use the available cloning technology to generate nucleic acid sequences or vectors suitable for being transduced into the cells.

Preferably, nucleic acid sequences encoding Ikzf2 protein and Atoh1 protein are provided as a vector, preferably as an expression vector. Preferably, it can be provided as a gene therapy vector that is preferably suitable for transduction and expression in target cells, such as cochlea supporting cells. The vector can be viral or non-viral (such as a plasmid). Viral vectors include those derived from adenovirus, adeno-associated virus (AAV) including mutant forms, retrovirus, lentivirus, herpesvirus, cowpox virus, MMLV, GaLV, simian immunodeficiency virus (SIV), HIV, poxvirus and SV40. Preferably, the viral vector is replication defective, or it can be replication deficient, which can be replicable, or conditionally replicable. The viral vector can usually maintain an extracellular state without integrating into the genome of the target cell. The preferred viral vector used to introduce Ikzf2 protein and Atoh1 protein to target cells is an AAV vector. Using specific AAV serum type (AAV serum type 2 to AAV serum type 12) or any modified version of any one of these serum types can achieve selective targeting. In the preferred embodiment of the present invention, the AAV vector is preferably AAV7m8, AAV-anc80, or AAV-ie.

The viral vector can be modified to delete any non-necessary sequence. For example, in AAV, the virus can be modified to delete all or part of the IX gene, Ela and/or Elb genes. For wild-type AAVs, there is no auxiliary virus such as adenovirus, and the replication is very inefficient. For the recombinant adeno-associated virus, preferably, the replication gene and the capsid gene are provided in trans form (in the pRep/Cap plasmid), and only the 2ITR of the AAV genome is retained and packaged into the virus body, while the required adenovirus gene is provided by the adenovirus or another plasmid. Similar modifications can also be made for lentiviral vectors.

The viral vector has the ability to enter cells. However, non-viral vectors such as plasmids can be combined with reagents to facilitate the uptake of viral vectors by target cells. Such reagents include polycation agents. Optionally, the delivery system, such as a liposome-based delivery system, can be used. The vectors used in the present invention preferably are suitable for being used in vivo or in vitro, and preferably suitable for being used in humans.

The vector will preferably contain one or more regulating sequences to guide the expression of nucleic acid sequences in target cells. The regulating sequence may include a promoter, an enhancer, a transcriptional termination signal, a polyadenylation sequence, an origin of replication, a nucleic acid restriction, and homologous recombination site that can be operably linked to the nucleic acid sequence. The vector may also include selective marks, such as determining the expression of the vector in the growth system (such as a bacterial cell) or in the target cells.

“Operably link” means that a nucleic acid sequence correlates with its operably connected sequence in the function, so that they are linked in a way that affects each other's expression or function. For example, a nucleic acid sequence that is operably linked to a promoter will have expression patterns influenced by the promoter.

The promoter mediates the expression of the nucleic acid sequence connected to it. The promoter may be constitutive or inducible. The promoter can guide the general expression of genes in cochlear cells, or the specific expression in cochlear cells. In the latter case, the promoter can guide specific expression of cell types, such as cochlea supporting cells (SC) (including pillar cells (PCs) and Deiters cells (DCs)). The appropriate promoter will be known to those skilled in this art. For example, the appropriate promoter may be selected from the group consisting of: L7, thy-1, recovered protein, calcium binding protein, human CMV, GAD-67, chicken muscle protein, CAG and CBA, etc. In addition, using cell specific promoters can achieve gene expression targeting specific cells.

An example of a suitable promoter is the immediate early cytomegalovirus (CMV) promoter sequence. The promoter sequence is a strongly constitutive promoter sequence capable of driving high-level expression of any polynucleotide sequence operably linked to it. Another example of a suitable promoter is the extension growth factor-1α (EF-1α). However, other constitutive promoter sequences can also be used, including but not limited to early promoter of simian virus 40 (SV40), mouse breast cancer virus (MMTV), human immunodeficiency virus (HIV) long-terminal repeat (LTR) promoter, MoMuLV promoter, avian leukemia virus promoter, immediate early promoter of Epstein-Barr virus, Ruth's sarcoma virus promoter, and human gene promoter, such as, but not limited to, an actin promoter, a myosin promoter, a heme promoter, and a creatine kinase promoter. Further, the present invention should not be limited to the use of constitutive promoters. Inducible promoters are also considered as part of the present invention. The use of inducible promoters provides a molecular switch that can turn on expression of a polynucleotide sequence operably linked to an inducible promoter when such expression is desired, or turn off expression when the expression is undesirable. Examples of inducible promoters include, but are not limited to, a metallothionein promoter, a glucocorticoid promoter, a progesterone promoter, and a tetracycline promoter.

Many expression vectors can be applied to the expression of Ikzf protein and Atoh1 protein in mammalian cells (preferably human cells, more preferably human cochlear supporting cells). An adeno-associated virus (AAV) vector is preferably used in the present invention as the expression vector.

The present invention also provides a host cell to express Ikzf2 protein and Atoh1 protein. Preferably, the host cells are mammalian cells (preferably human cells, more preferably human cochlear supporting cells), thereby increasing the expression of Ikzf2 protein and Atoh1 protein.

Pharmaceutical Preparation and Composition

The present invention provides a pharmaceutical preparation or composition that contains the combination of active ingredients of the second aspect of the present invention, or the vector of the third aspect of the present invention, or the host cell of the fourth aspect of the present invention, and (b) a pharmaceutical acceptable carrier or excipient.

In another preferred embodiment, the pharmaceutical preparation is used to treat hearing impairment.

In another preferred embodiment, the pharmaceutical preparation is used to treat the degeneration and/or damage of cochlea outer hair cells.

The “active ingredient” in the pharmaceutical preparation of the present invention refers to the vector described in the present invention, such as a viral vector (including an adeno-associated virus vector). The “active ingredient”, preparation and/or composition described in the present invention can be used to treat hearing impairment. “Safe and effective amount” means that the amount of active ingredient is sufficient to significantly improve the condition or symptom without serious side effects. “Pharmaceutically acceptable carrier or excipient” refers to: one or more compatible solid or liquid fillers or gel materials, which are suitable for human use, and must be of sufficient purity and low toxicity. “Compatibility” herein means that each component in the composition can be mixed with the active ingredient of the invention and among them without significantly reducing the efficacy of the active ingredient.

The composition may be a liquid or solid, such as a powder, gel or paste. Preferably, the composition is a liquid, and preferably an injectable liquid. The appropriate excipient will be known to those skilled in this art.

In the present invention, the vector can be administrated directly to the ear or cochlea of a subject. In any one administration mode, the vector is preferably provided as an injectable liquid. Preferably, the injectable liquid is provided as a capsule or syringe.

In the present invention, the host cells of the present invention can be implanted into the cochlea of a subject for administration. Preferably, the host cells are provided as an injectable liquid.

Some examples of pharmaceutically acceptable carrier include cellulose and its derivatives (such as sodium carboxymethyl cellulose, sodium ethyl cellulose, cellulose acetate, etc.), gelatin, talc, solid lubricant (such as stearic acid, magnesium stearate), calcium sulfate, vegetable oil (such as soybean oil, sesame oil, peanut oil, olive oil, etc.), polyol (such as propylene glycol, glycerol, mannitol, sorbitol, etc.), emulsifier (such as Tween®), wetting agent (such as sodium dodecyl sulfate), colorant, flavor, stabilizer, antioxidant, preservative, pyrogen free water, and the like.

The composition may comprise physiologically acceptable sterile aqueous or non-aqueous solutions, dispersions, suspensions or emulsions, and sterile powders for reconstitution into sterile injectable solutions or dispersions. Suitable aqueous and non-aqueous carriers, diluents, solvents or excipients include water, ethanol, polyol, and suitable mixtures thereof.

Therapeutic Method

The present invention provides a method of regenerating cochlea outer hair cells, which comprises introducing the nucleotide sequences encoding Ikzf2 protein and Atoh1 protein into the cochlea. The method may include administrating a vector containing nucleotide sequences encoding Ikzf2 protein and Atoh1 protein to supporting cells in the cochlea.

The present invention provides a pharmaceutical preparation or composition used in the method of treating the hearing impairment by regenerating cochlea outer hair cells, wherein the pharmaceutical preparation or composition comprises the vector containing nucleotide sequences encoding Ikzf2 protein and Atoh1 protein, or the host cells containing the vector, and a pharmaceutically acceptable carrier or excipient. The pharmaceutical preparation or composition of the present invention may be administered alone or in combination with other therapeutic drugs (which, for example is prepared in the same pharmaceutical composition).

The present invention also provides a method of treating hearing impairment, which comprises administrating the pharmaceutical preparation or composition of the present invention to a subject. The hearing impairment is related to the degeneration and/or damage of cochlea outer hair cells. The method comprises directly administrating the vector containing nucleotide sequences encoding Ikzf2 protein and Atoh1 protein of the present invention to the ear (cochlea) of the subject in need thereof. The method further comprises directly administrating the host cells of the present invention to the cochlea of the subject in need thereof.

As used herein, “regeneration of cochlea outer hair cells” means that cells that previously did not have the capacity of cochlea outer hair cells or whose capacity of cochlea outer hair cells has been completely or partially degraded become cells with the characteristics and functions of cochlea outer hair cell after expressing foreign nucleic acid sequences encoding Ikzf2 protein and Atoh1 protein. Such cells can be called transformed cells herein, or “cochlea outer hair cell-like cells (OHC-like cells)”, because they contain unnatural nucleic acids. Preferably, the transformed cochlea outer hair cells exhibit some or all of the capacity of the natural cochlea outer hair cells. Preferably, the transformed cells exhibit at least the same or substantially the same auditory ability as the natural cochlea outer hair cells. Preferably, the transformed cells exhibit higher auditory ability than the diseased or degenerating natural cochlea outer hair cells. Therefore, the transformed cells will preferably have increased cochlea outer hair cells, compared with degenerated or diseased cells from the same source which are maintained under the same conditions, and untreated. Transformed cells can be distinguished from natural cells by the presence of exogenous nucleic acids within them.

Compared with prior art, main advantages of the present invention comprise:
(1) The present inventor co-expressed Ikzf2 protein and Atoh1 protein heterotopic in cochlea supporting cells, thereby transforming them into OHC-like cells. The transformed OHC-like cells have a tissue form and genetic characteristic similar to natural OHCs, and have the same OHC function.
(2) After OHC injury, the method of the present invention can produce OHC-like cells quickly and efficiently.
(3) The method of the present invention provides a new idea of gene therapy for hearing disorders and hearing impairment.

The present invention is further explained below in conjunction with specific examples. It should be understood that these examples are only for illustrating the present invention and not intend to limit the scope of the present invention. The conditions of the experimental methods not specifically indicated in the following examples are usually in accordance with conventional conditions as described in Sambrook et al., Molecular Cloning: A Laboratory Manual (New York: Cold Spring Harbor Laboratory Press, 1989), or according to the conditions recommended by the manufacturer. Unless otherwise stated, percentages and parts are calculated by weight.

Methods and Materials:

(1) Rosa26-CAG-LSL-Ikzf2/+ and Prestin-DTR/+ Targeted Knock-In Mouse Strains were Constructed by CRISPR/Cas9 Method.

At the same time, Rosa26 sgRNA (5′-actccagttttctagagaga-3′, SEQ ID No: 5), donor DNA and Cas9 mRNA were injected into Zygote at one cell stage at the same time to construct Rosa26-CAG-LSL-Ikzf2/+ targeted knock-in mice. In the same way, the Prestin-P2A-DTR/+(Prestin-DTR/+) targeted knock-in mouse strain was constructed (Prestin (SLC26A5), and its sgRNA is

| (SEQ ID NO: 6) | |
|---|---|
| | 5′-CGAGGCATAAAGGCCCTGTA-3′ |

The correct mice in F0 were screened through the junction PCR method, and then the potential correct F0 mice were matched with wild-type C57BL/6 mice to get F1 mice. Firstly, the tail of F1 mice was identified by junction PCR to identify potential correct mice. Then, southern blotting experiments were conducted to exclude mice with random insertion in F1 based on the method described in (Li et al., 2018). All mice are reproduced and raised in the SPF level mouse house. All operations are conducted under the guidance of Institute of Neuroscience and the Center for Excellence in Brain Science and Intelligent Technology Innovation of the Chinese Academy of Sciences.

(2) Sample Treatment, Immunohistochemistry and Cell Counting

Adult mice were subjected to cardiac perfusion by using 1×PBS and 4% PFA to completely remove blood from the inner ear and pre-fixation. Then the inner ear was carefully dissected and put in a tube containing 4% PFA and fixed at 4° C. overnight. The next day, the inner ear was washed with 1×PBS for 10 minutes×3 times, and then 120 mm EDTA was used to decalcify the inner ear for 2 days until the inner ear became soft. Then the entire cochlea epithelium was dissected for immunohistochemistry.

The primary antibodies used are shown in the following table:

Antibody information table

| Antibody name | Antibody information |
|---|---|
| anti-HA | rat, 1:200, 11867423001, Roche |
| anti-Prestin | goat, 1:1000, SC-22692, Santa Cruz |
| anti-Myo6 | rabbit, 1:500, 25-6791, Proteus Bioscience |
| anti-Myo7a | rabbit, 1:500, 25-6791, Proteus Bioscience |
| anti-Sox2 | goat, 1:500, sc-17320, Santa Cruz |
| anti-Insm1 | guinea pig, 1:6000, Presented by Dr. Jia |
| | Shiqi, Jinan University, Guangzhou, China, |
| | and Dr. Carmen Birchmeier, Max Delbruck |
| | Molecular Medical Center, Berlin, Germany |
| anti-Parvalbumin | mouse, 1:500, P3088, Sigma |
| anti-Rbm24 | rabbit, 1:500, 18178-1-AP, Proteintech |
| anti-Calbindin | rabbit, 1:500, C9848, Sigma |
| anti-vGlut3 | rabbit, 1:500, 135203, Synaptic System |

Cochlea tissue was stained with Hoechst33342 (1:1000, 62249, Thermo Scientific) dissolved in PBST for observation of cell nucleus, and the section was sealed with Prolong gold anti fade medium (P36930, Thermo Scientific). Nikon C2, TiE-A1 and NiE-A1 plus confocal microscope was used to take pictures.

During dissection, each cochlea was divided into three segments and photographed under a 10× objective lens. Then, a line was drawn between the inner and outer hair using a software to measure the length of the cochlea, and the cochlea was evenly divided into three segments: basal, middle, and apical. In order to observe the death of outer hair cells in the Prestin DTR/+ mouse model, each segment was randomly photographed at two locations under the 60× objective lens, the counting results of the two locations were averaged, and inner hair cells were used as a reference, because DT did not kill inner hair cells. For the counting of Prestin in the inner hair cells of Atoh1CreeER+; Rosa26-CAG-LSL-Ikzf2/+ mice, and the signals of Tdtomato+/HA+ cells, OHC-like cells and hair cells in the early neonatal period in the Fgfr3-Atoh1-Ikzf2 and Fgfr3-Atoh1-Ikzf2-DTR mouse models, the whole cochlea was photographed in a 60× jigsaw pattern for more accurate counting. Graphpad Prism 6.0 was used to make statistical diagrams, and the Student's T test and Mean±SEM were used for statistical analysis of data.

(3) Single Cell Sequencing and Bioinformatics Analysis

Tdtomato+ cells were used in 3 mouse models: 1) Prestin-CreER/+; Ai9/+ mice were administrated with Tamoxifen at P20 and P21, and cells were picked at P30 (Fang et al., 2012); 2) Fgfr3-iCreER+; Ai9/+ mice were administrated with Tamoxifen at P30 and P31, and supporting cells were picked at P60 (primarily PCs and DCs), according to previous reports (Liu et al., 2012a; Liu et al., 2012b); 3) Fgfr3-Atoh1-Ikzf2-DTR mice were administrated with Tamoxifen at P30 and P31, with DT at P36, and cells were picked at P60.

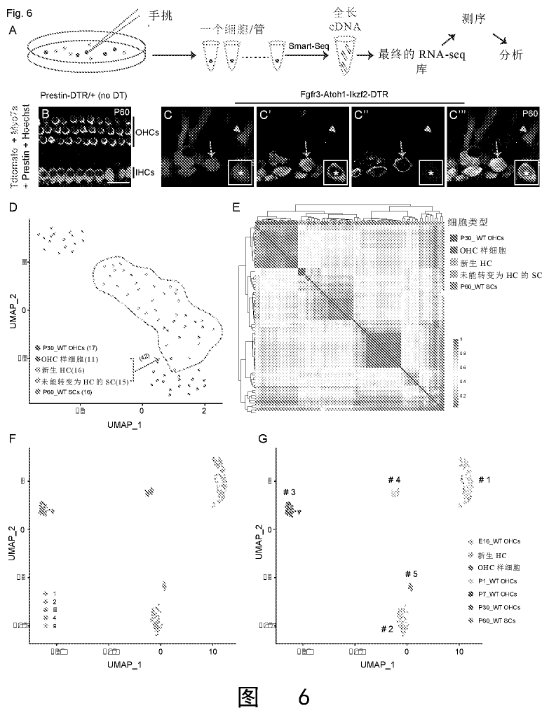

Tdomato+cells were picked out by hand under the stereomicroscope, including 17 P30 wild-type outer hair cells, 16 P60 wild-type supporting cells, and 42 cells in the Fgfr3-Atoh1-Ikzf2-DTR model (FIG. 6D), which were subjected to reverse transcription immediately and cDNA amplification with smart-Seq HT kit (Cat #634437, Takara). TruePrep DNA Library Prep Kit V2 (Cat #TD503, Vazyme) and TruePrep Index Kit V2 (Cat #TD202, Vazyme) was used for quality inspection. Finally, the cDNA library was paired and sequenced using the Illumina Novaseq platform, with a sequencing depth of 4G for each library.

FastQC (v0.11.9) and trimmomatic (v0.39) were used for the quality inspection on raw sequencing data. Approximately 70-80% of reads of the mouse reference genes were covered with high quality, using the default parameters of Hisat2 (v2.1.0). The original data were calculated through HTSeq (v0.10.0). Default parameters of Stringtie (v1.3.5) were used to estimate the level of gene expression. The abundance of genes was presented in terms of transcripts per million (TPM). R package “DESeq2” (p.adj<0.05, absolute value of (log 2 Fold Change)>2) was used to analyze the expression of different genes. DEGs was used for the enrichment process of bioanalysis (p<0.05, calibrated with bonferroni corrections), while annotating, visualizing, and integrating the database. The original data Geo (Gene Expression Omnibus) library of all single cell sequencing of the present invention can be obtained by search code: GSE161156.

Seurat (R package v3.0) software was used to analyze the data of wild type outer hair cells of E16, P1 and P7 in the literature (Kolla et al., 2020). In order to more accurately compare the transcriptome data of 10× and samrt-seq sequencing method analysis, they were first integrated with the function of “FindIntegrationAnchors” (k.filter=30). Then, the component calculation was performed using the “RunPCA” function, and the top 20 principal components were subjected to dimensionality reduction analysis by using “RunTSNE” and “RunUMAP”. A unsupervised cluster analysis was performed with the function of “FindClusters” (resolution=0.5). In addition, in order to more accurately analyze the progenitor cells of (E16, E17 and P7) hair cells and (E14) hair cells in single-cell sequencing, the set standard is more strict than the traditional standard. Among them, the expression of Insm1, Myo6 and Atoh1 was greater than 0 in E16 wild-type outer hair cells, and Bcl11b, Myo6, Myo7a and Atoh1 were greater than 0 in P1 wild-type outer hair cells. In wild type outer hair cells of P7, Prestin (Slc26a5), Myo6, Ocm, and Ikzf2 were greater than 0. Monocle (R package v2.0) package was used for trajectory analysis. The “Importcds” function was used to introduce the pre-processed Seurat data into Monocle through the “ImportCDS” function. In the outer hair cells of three ages, the genes with significant changes were identified through different expression analysis. By using the “orderCells” function in Monocle, cells were presented along a hypothetical axis.

(4) Auditory Brainstem Response (ABR)

The auditory brainstem response of mice was tested using the previous method (Li et al., 2018). Prestin-DTR/+ and Fgfr3-Atoh1-Ikzf2-DTR mice were subjected to hearing tests at frequencies of 4 k Hz, 5.6 k Hz, 8 k Hz, 11.3 k Hz, 16 k Hz, 22.6 k Hz, and 32 k Hz, respectively, at P42 and P60 (with or without DT treatment at P36). The data was statistically analyzed using Student's t test.

(5) Tamoxifen and Diphtheria Toxin

Tamoxifen (Cat #T5648, Sigma) was dissolved in corn oil (Cat #C8267, Sigma) for subsequent experiments, and was injected into the abdominal cavity at doses of 3 mg/40 g (P0, P1) or 9 mg/40 g (P20, P21, and P30, P31) based on body weight. Diphtheria toxin (Cat #D0564, Sigma) was dissolved at 0.9% NaCl, and was injected into the abdominal cavity at P36 according to the dose of 20 ng/g.

(6) Scan Electron Microscope (SEM)

SEM experiments can be conducted based on a previous method (Parker et al., 2016). Firstly, we used tweezers to make a hole at the top of the cochlea, and then slowly injected 0.9% NaCl (Cat #10019318, Sinopharm Chemical Agent Co, Ltd) into the circular window to flush the cochlea. Then, we pre-fixed the cochlea with 2.5% glutaraldehyde (Cat #G5882, Sigma), and exposed as many of the Corti organs as possible with tweezers for sufficient fixation. Finally, we placed the cochlea in 2.5% glutaraldehyde overnight at 4° C. The next day, the inner ear was rinsed for 3 times with 1×PBS and decalcified with 10% EDTA (Cat #ST066, Beyotime) for 1 day. Then, the cochlea was dissected and fixed with 1% osmium tetroxide (OsO4, Cat #18451, Tedpella) for 1 hour, washed for 6 times with ddH2O, fixed with TCH (thiocarbohydyazide, Cat #88535, Sigma) for 30 minutes, washed 6 for times with ddH2O, fixed with 1% osmium tetroxide for 1 hour, and washed for 6 times with ddH2O. Afterwards, different concentrations of ethanol (30%, 50%, 75%, 80%, 95%, Cat #10009259, Sinopharm Chemical Agent Co, Ltd) were used for gradient dehydration of the cochlea samples at 4° C. for 30 minutes in each step. Then dehydration was performed for 3 times in 100% ethanol for 30 minutes each time. Afterwards, critical point drying was performed on the sample (model: EM CPD300, Leica). Then the cochlea was pasted face up onto a conductive adhesive and the sample was sprayed with gold (model: Q150T ES, Quorum). Finally, the sample was photographed using a scanning electron microscope (model: GeminiSEM 300, ZEISS).

### Example 1

After Ectopic Overexpression of Ikzf2 in the Inner Hair Cells, the Inner Hair Cells Began to Express Prestin

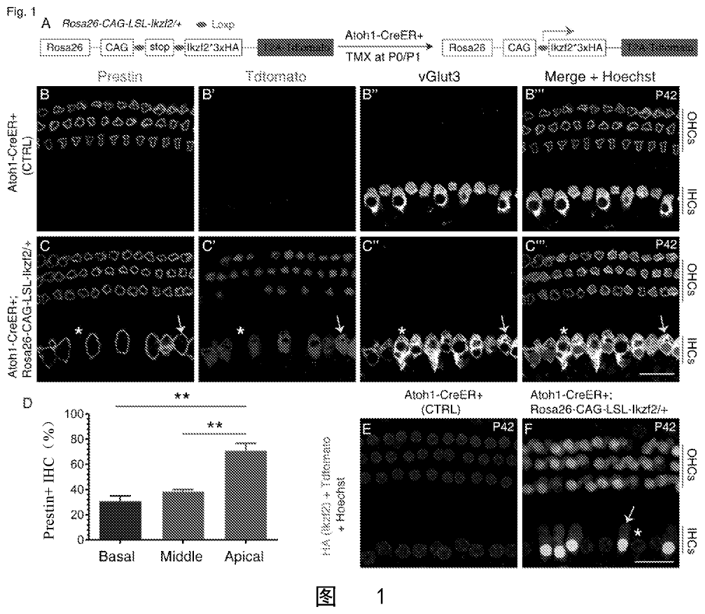

In the wild-type mice, both of Ikzf2 and Prestin are only expressed in the cochlea outer hair cells, and the inner hair cells never express Ikf2 and Prestin. The Rosa26-CAG-LSL-Ikzf2/+ gene-targeted mice were constructed to overexpress Ikzf2. FIG. 1A shows the ectopic expression principle of Ikzf2. When Ikzf2 was expressed, the HA tag fused with Ikzf2 and the Tdtomato immediately adjacent it were also expressed, causing cells expressing Ikzf2 to emit red fluorescence. After tamoxifen was injected into mice, compared with control mice (FIG. 1B-B′″), when Ikzf2 was forcibly overexpressed in inner hair cell, prestin was also ectopic-expressed in inner hair cell (FIG. 1C-C′″). Quantitative analysis of Prestin positive inner hair cells showed that there were more Prestin positive inner hair cells in Apical turn than in Middle and Basal turn (FIG. 1D). After verification, only the experimental group (Figure F), instead of the control group (Figure E), expressed HA/Tdtomato. All Tdtomato positive cells expressed HA (Ikzf2), and vice versa.

### Example 2

Overexpression of Both of Atoh1 and Ikzf2 in PCs and DCs of Adult Mice can Generate a Small Amount of OHC-Like Cells

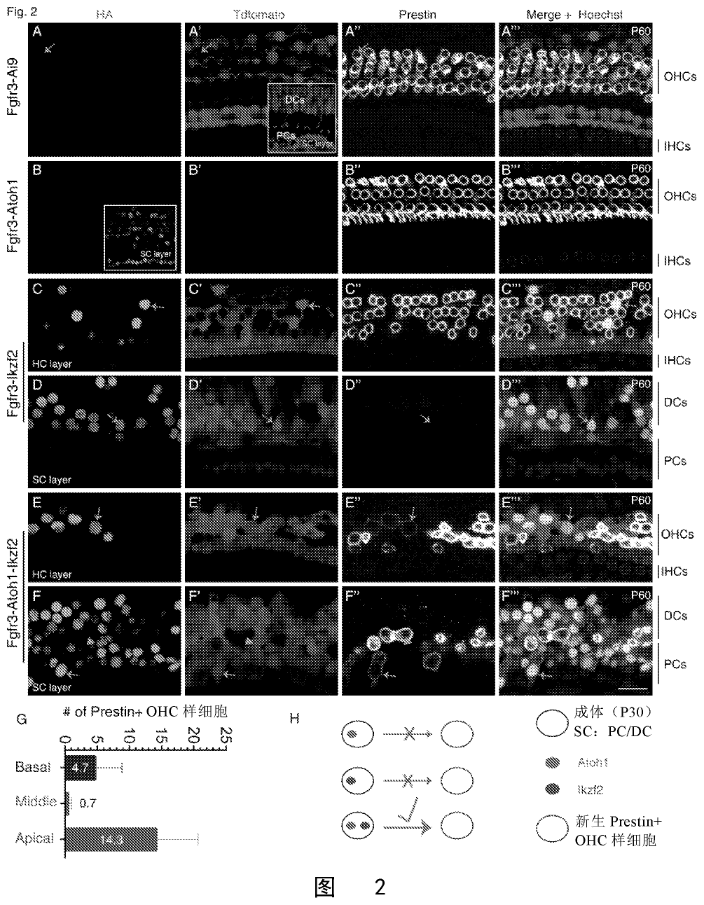

The effects of different treatment methods on the production of OHC-like cells were compared. That is, three different treatments were made in the adult cochlea supporting cells: expressing Atoh1 alone, expressing Ikzf2 alone, and expressing Atoh1 and Ikzf2 at the same time. Compared with the control group (FIG. 2A-A′″), overexpressing Atoh1 alone (FIG. 2B-B′″) or overexpressing Ikzf2 alone (FIG. 2C-C′″ and FIG. 2D-D′″) could not produce new Prestin+ outer hair cells. However, overexpressing Atoh1 and Ikzf2 at the same time (FIG. 2E-E′″ and FIG. 2F-F′″) could produce a small amount of new Prestin+ outer hair cells. Quantitative analysis of OHC-like cells in mice overexpressing Atoh1 and Ikzf2 at the same time (Fgfr3-Atoh1-Ikzf2) (FIG. 2G) revealed that new cells were visible throughout the cochlea, however the number was small and varied significantly. Summing up, for the three different reprogramming methods, it was found that only by simultaneously activating Atoh1 and Ikzf2 can OHC-like cells be produced (FIG. 2H).

### Example 3

Specific Damage of Outer Hair Cells by Genetic and Pharmacological Methods

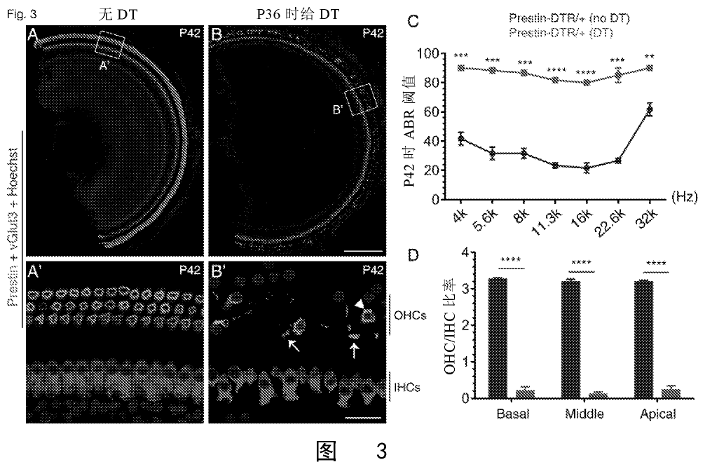

In order to simulate human hearing impairment model, the Prestin-P2A-DTR/+ knock-in mice were constructed. After the adult (P36) mice were treated with diphtheria toxin (DT), the cochlea outer hair cells were specifically killed (FIG. 3A-A′ and FIG. 3B-B′). Statistical analysis shows the death of more than 90% of outer hair cells (Note: the inner hair cells were normal). In addition, after DT treatment, the hearing threshold of the cochlea increased significantly (FIG. 3C). The statistical analysis of the OHC/IHC ratio of the control group and the experimental group showed that the ratio of the experimental group was significantly lower than that of the control group (FIG. 3D). The above results showed that the hearing impairment model was successfully constructed.

### Example 4

After Injury to In Situ Outer Hair Cells, the Reprogramming Efficiency of Overexpression of Atoh1 and Ikzf2 in Adult Supporting Cells can be Promoted

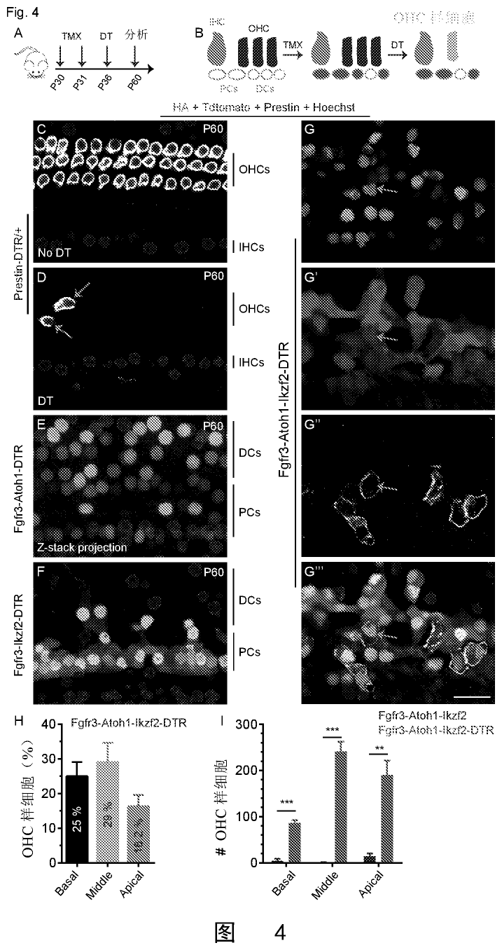

The hearing impairment model was established by genetic and pharmacological methods, and the specific killing on adult outer hair cells were achieved. Then, in adult supporting cells of hearing impairment model mice, Atoh1 was overexpressed alone (FIG. 4E), Ikzf2 was overexpressed alone (FIG. 4F), or both Atoh1 and Ikzf2 were overexpressed simultaneously (FIG. 4G-G′″). FIG. 4A and FIG. 4B show the process of model construction. The experimental results show that only overexpressing Atoh1 and Ikzf2 simultaneously can generate a large number (400-600) of new Prestin+ OHC-like cells. At the same time, the quantitative analysis of the number of OHC-like cells in the Fgfr3-Atoh1-Ikzf2-DTR mice (FIG. 4H), and the comparison of the number of OHC-like cells in the Fgfr3-Atoh1-Ikzf2-DTR and Fgfr3-Atoh1-Ikzf2 mouse models (without destruction of in situ hair cells) were conducted. It was found that Fgfr3-Atoh1-Ikzf2-DTR has more OHC-like cells than Fgfr3-Atoh1-Ikzf2. The above results indicate that killing endogenous outer hair cells can significantly improve the efficiency of Atoh1 and Ikzf2 transforming supporting cells into new outer hair cells.

### Example 5

OHC-Like Cells Exhibit Irregular Ciliary Bundles

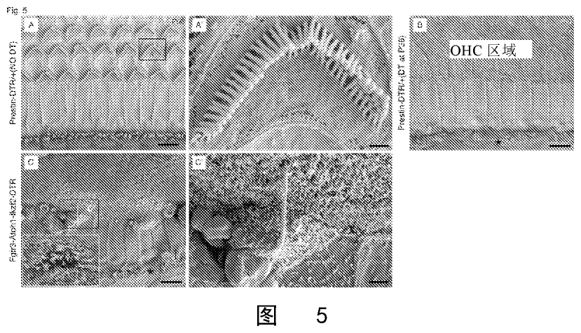

Detailed analysis of the auditory epithelium of the cochlea of three different genotypes of mice was conducted using ultra-high scanning electron microscopy. There are ‘V’ or ‘W’ shaped cilia on the top of the outer hair cell of wild-type mice (FIG. 5A-A′). The ultrastructure of cilia is very important for outer hair cell to perceive sound. Cilia structure disappeared in the outer hair cell killing model (FIG. 5B), but reappeared in the model of adult supporting cells overexpressing Atoh1 and Ikzf2 (FIG. 5C-C′). This result shows that new hair cells (ie, OHC-like cells) have cilia structure.

### Example 6

OHC-Like Cells are Similar to Wild-Type Outer Hair Cells of P1

The wild-type cochlea supporting cells and outer hair cells were marked red by genetic means, then the cochlea tissue was digested, and single-cell transcriptome analysis was performed by manual sorting (FIG. 6A). At the same time, using a similar method, supporting cells overexpressing Atoh1 and Ikzf2 were also selected for single-cell transcriptome analysis (FIG. 6A). Myo7a and Prestin were used for immunohistochemistry (FIG. 6B-C′″), it was found that the supporting cells expressing Atoh1 and Ikzf2 eventually had three cell fates: 1) not transforming into hair cells (triangle); 2) transforming into immature hair cells (asterisk); 3) transforming into newly generated Prestin+ OHC-like cells (the type of concern of the present invention) (arrow). UMAP analysis show that the newly generated cells are closer to the adult outer hair cells, but not completely coincident (FIG. 6D and FIG. 6E). For further analysis, by comparing the transcriptome data of hair cells in different periods recently published in Nature Communications in 2020, it is found that the newly generated Prestin+ outer hair cells are the most similar to the wild-type outer hair cell on the first day after birth (P1) (FIG. 6F and FIG. 6G).

All references mentioned in the present application are incorporated by reference herein, as though individually incorporated by reference. In addition, it should be understood that after reading the above teaching content of the present invention, various changes or modifications may be made by those skilled in the art, and these equivalents also fall within the scope as defined by the appended claims of the present application.

1. A method of (i) regenerating cochlea outer hair cells, and/or (ii) treating or preventing hearing impairment, comprising using a combination of active ingredients;
wherein the combination of active ingredients comprises:
(a) Ikzf2 protein, a coding sequence thereof, or a promoting agent thereof, or a combination thereof; and
(b) Atoh1 protein, a coding sequence thereof, or a promoting agent thereof, or a combination thereof.

2. The method of claim 1, wherein the hearing impairment is related to the degeneration and/or damage of cochlea outer hair cells (OHCs).

3. A combination of active ingredients that can be used to regenerate cochlea outer hair cells, comprising:
(a) Ikzf2 protein, a coding sequence thereof, or a promoting agent thereof, or a combination thereof; and
(b) Atoh1 protein, a coding sequence thereof, or a promoting agent thereof, or a combination thereof.

4. An expression vector comprising:
(Z1) a first expression vector containing a first expression cassette used to express Ikzf2 protein;
(Z1) a second expression vector containing a second expression cassette used to express Atoh1 protein;
wherein the first expression vector and the second expression vector are the same vector or different vectors.

5. A host cell comprising the expression vector of claim 4.

6. The host cell of claim 5, wherein the host cells are selected from the group consisting of cochlea supporting cells (SCs), including pillar cells (PCs), Deiters cells (DCs) and a combination thereof.

7. A pharmaceutical preparation containing (a) the combination of active ingredients of claim 3, and (b) a pharmaceutically acceptable carrier or excipient.

8. (canceled)

9. A method of regenerating cochlea outer hair cells, comprising transducing the combination of active ingredients of claim 3 into cochlea supporting cells.

10. A method of (i) promoting regeneration of cochlea outer hair cells, and/or (ii) auxiliary treatment or prevention of hearing impairment, comprising using an active ingredient;
wherein the active ingredient comprises: Ikzf2 protein, a coding sequence thereof, or a promoting agent thereof, or a combination thereof.

11. A pharmaceutical preparation containing (a) the expression vector of claim 4, and (b) a pharmaceutically acceptable carrier or excipient.

12. A pharmaceutical preparation containing (a) the host cell of claim 5, and (b) a pharmaceutically acceptable carrier or excipient.

13. A method of regenerating cochlea outer hair cells, comprising transducing the expression vector of claim 4 into cochlea supporting cells.

14. A method of treating or preventing hearing impairment, comprising administrating the expression vector of claim 4 to a subject in need thereof.

15. A method of treating or preventing hearing impairment, comprising administrating the host cell of claim 5 to a subject in need thereof.


SEQUENCE LISTING (8 sequences)

SEQ ID NO: 1 - protein, 526 aa, Homo sapiens [sequence omitted]

SEQ ID NO: 2 - protein, 354 aa, Homo sapiens
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENSKTSPRSHRSDGEFSPHSHYSDSDEAS

SEQ ID NO: 3 - DNA, 1581 bp, Homo sapiens
atggaaacagaggctattgatggctatataacgtgtgacaatgagctttcacccgaaagggagcactccaatatggcaattgacctcacctcaagcacacccaatggacagcatgcctcaccaagtcacatgacaagcacaaattcagtaaagctagaaatgcagagtgatgaagagtgtgacaggaaacccctgagccgtgaagatgagatcaggggccatgatgagggtagcagcctagaagaacccctaattgagagcagcgaggtggctgacaacaggaaagtccaggagcttcaaggcgagggaggaatccggcttccgaatggtaaactgaaatgtgacgtctgtggcatggtttgcattgggcccaatgtgcttatggtacataaaaggagtcacactggtgaacgccccttccactgtaaccagtgtggagcttcttttactcagaagggcaaccttctgagacacataaagttacactctggagagaagccgttcaaatgtcctttctgtagctacgcctgtagaagaagggacgccctcacaggacacctcaggacccattctgtgggtaaacctcacaagtgcaactactgtggacgaagctacaagcagcgcagttcactggaggagcacaaggaacgctgccacaactatctccagaatgtcagcatggaggctgctgggcaggtcatgagtcaccatgtacctcctatggaagattgtaaggaacaagagcctattatggacaacaatatttctctggtgccttttgagagacctgctgtcatagagaagctcacggggaatatgggaaaacgtaaaagctccactccacaaaagtttgtgggggaaaagctcatgcgattcagctacccagatattcactttgatatgaacttaacatatgagaaggaggctgagctgatgcagtctcatatgatggaccaagccatcaacaatgcaatcacctaccttggagctgaggcccttcaccctctgatgcagcacccgccaagcacaatcgctgaagtggccccagttataagctcagcttattctcaggtctatcatccaaataggatagaaagacccattagcagggaaactgctgatagtcatgaaaacaacatggatggccccatctctctcatcagaccaaagagtcgaccccaggaaagagaggcctctcccagcaatagctgcctggattccactgactcagaaagcagccatgatgaccaccagtcctaccaaggacaccctgccttaaatcccaagaggaaacaaagcccagcttacatgaaggaggatgtcaaagctttggatactaccaaggctcctaagggctctctgaaggacatctacaaggtcttcaatggagaaggagaacagattagggccttcaagtgtgagcactgccgagtccttttcctagaccatgtcatgtacaccattcacatgggttgccatggctaccgggacccactggaatgcaacatctgtggctacagaagccaggaccgttatgagttttcatcacacattgttcgaggggagcacacattccactag

SEQ ID NO: 4 - DNA, 1065 bp, Homo sapiens
atgtcccgcctgctgcatgcagaagagtgggctgaagtgaaggagttgggagaccaccatcgccagccccagccgcatcatctcccgcaaccgccgccgccgccgcagccacctgcaactttgcaggcgagagagcatcccgtctacccgcctgagctgtccctcctggacagcaccgacccacgcgcctggctggctcccactttgcagggcatctgcacggcacgcgccgcccagtatttgctacattccccggagctgggtgcctcagaggccgctgcgccccgggacgaggtggacggccggggggagctggtaaggaggagcagcggcggtgccagcagcagcaagagccccgggccggtgaaagtgcgggaacagctgtgcaagctgaaaggcggggtggtggtagacgagctgggctgcagccgccaacgggccccttccagcaaacaggtgaatggggtgcagaagcagagacggctagcagccaacgccagggagcggcgcaggatgcatgggctgaaccacgccttcgaccagctgcgcaatgttatcccgtcgttcaacaacgacaagaagctgtccaaatatgagaccctgcagatggcccaaatctacatcaacgccttgtccgagctgctacaaacgcccagcggaggggaacagccaccgccgcctccagcctcctgcaaaagcgaccaccaccaccttcgcaccgcggcctcctatgaagggggcgcgggcaacgcgaccgcagctggggctcagcaggcttccggagggagccagcggccgaccccgcccgggagttgccggactcgcttctcagccccagcttctgcgggagggtactcggtgcagctggacgctctgcacttctcgactttcgaggacagcgccctgacagcgatgatggcgcaaaagaatttgtctccttctctccccgggagcatcttgcagccagtgcaggaggaaaacagcaaaacttcgcctcggtcccacagaagcgacggggaattttccccccattcccattacagtgactcggatgaggcaagttag

SEQ ID NO: 5 - protein, 526 aa, Mus musculus
METDAIDGYITCDNELSPEGEHANMAIDLTSSTPNGQHASPSHMTSTNSVKLEMQSDEECDRQPLSREDEIRGHDEGSSLEEPLIESSEVADNRKVQDLQGEGGIRLPNGKLKCDVCGMVCIGPNVLMVHKRSHTGERPFHCNQCGASFTQKGNLLRHIKLHSGEKPFKCPFCSYACRRRDALTGHLRTHSVGKPHKCNYCGRSYKQRSSLEEHKERCHNYLQNVSMEAAGQVMSHHVPPMEDCKEQEPIMDNNISLVPFERPAVIEKLTANMGKRKSSTPQKFVGEKLMRFSYPDIHFDMNLTYEKEAELMQSHMMDQAINNAITYLGAEALHPLMQHAPSTIAEVAPVISSAYSQVYHPNRIERPISRETSDSHENNMDGPISLIRPKSRPQEREASPSNSCLDSTDSESSHDDRQSYQGNPALNPKRKQSPAYMKEDVKALDATKAPKGSLKDIYKVFNGEGEQIRAFKCEHCRVLFLDHVMYTIHMGCHGYRDPLECNICGYRSQDRYEFSSHIVRGEHTFH

SEQ ID NO: 6 - protein, 351 aa, Mus musculus
MSRLLHAEEWAEVKELGDHHRHPQPHHVPPLTPQPPATLQARDLPVYPAELSLLDSTDPRAWLTPTLQGLCTARAAQYLLHSPELGASEAAAPRDEADSQGELVRRSGCGGLSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPNVGEQPPPPTASCKNDHHHLRTASSYEGGAGASAVAGAQPAPGGGPRPTPPGPCRTRFSGPASSGGYSVQLDALHFPAFEDRALTAMMAQKDLSPSLPGGILQPVQEDNSKTSPRSHRSDGEFSPHSHYSDSDEAS

SEQ ID NO: 7 - DNA, 1581 bp, Mus musculus
atggaaacagacgcaattgatggctatataacatgtgacaatgagctttcacccgaaggggaacacgccaatatggccattgacctcacctcaagcacacccaatggacagcacgcctcgccaagtcacatgacaagcacaaattctgtaaagctggaaatgcagagtgatgaagagtgtgacaggcagcccctgagccgtgaggatgagatcaggggccacgatgaggggagcagcctagaagaacccctaattgagagcagcgaggtggccgacaacaggaaagtccaggaccttcaaggcgagggaggaatccggcttccgaatggtaaactgaaatgtgacgtctgtggcatggtttgcattgggcccaatgtgcttatggtacataaaaggagtcacactggtgagcggcccttccactgtaaccagtgcggagcttcttttacccagaagggcaaccttctgagacacataaagttacactctggagagaagcccttcaaatgtcctttctgtagctatgcttgtagaagaagggacgctctcacaggacacctcaggacccattctgtgggtaaacctcacaagtgtaactactgtggccgaagctacaagcagcgcagctcactggaggaacacaaggaacgctgtcacaactatctccagaatgtcagcatggaggctgccgggcaggtcatgagtcaccatgtaccgcctatggaagattgtaaggaacaagagcctatcatggacaacaatatttctctggtgccttttgagagacctgctgtcatagagaagctcacggcaaatatgggaaagcgcaaaagctccactcctcagaagtttgtgggggaaaagcttatgcgattcagctacccagatattcattttgatatgaacttaacatatgagaaggaggctgagctgatgcagtctcatatgatggaccaagccatcaacaatgcaatcacctaccttggagctgaggcccttcaccctctgatgcagcatgcaccaagcacaatcgctgaggtggccccagttataagctcagcttattctcaggtctatcatccaaacaggatagaaagacccattagcagggaaacatctgatagtcacgaaaacaacatggatggccccatctctctcatcagaccaaagagtcgaccccaggaaagagaggcctcgcccagcaatagctgcctcgattctactgactcagaaagtagccatgatgaccgccagtcctaccaaggaaaccctgccttaaatcccaagaggaaacaaagcccagcttacatgaaggaggatgtcaaggctttggatgctaccaaggcccccaagggctctctgaaggacatctataaggttttcaatggagaaggagaacagataagggccttcaagtgtgagcactgccgagtcctttttctagaccatgtcatgtacaccattcacatgggttgccatggctaccgggacccactggaatgcaacatctgtggctacagaagccaggaccgctacgaattttcatcacacattgttcgaggggagcacacattccactag

SEQ ID NO: 8 - DNA, 1056 bp, Mus musculus
atgtcccgcctgctgcatgcagaagagtgggctgaggtaaaagagttgggggaccaccatcgccatccccagccgcaccacgtcccgccgctgacgccacagccacctgctaccctgcaggcgagagaccttcccgtctacccggcagaactgtccctcctggatagcaccgacccacgcgcctggctgactcccactttgcagggcctctgcacggcacgcgccgcccagtatctgctgcattctcccgagctgggtgcctccgaggccgcggcgccccgggacgaggctgacagccagggtgagctggtaaggagaagcggctgtggcggcctcagcaagagccccgggcccgtcaaagtacgggaacagctgtgcaagctgaagggtggggttgtagtggacgagcttggctgcagccgccagcgagccccttccagcaaacaggtgaatggggtacagaagcaaaggaggctggcagcaaacgcaagggaacggcgcaggatgcacgggctgaaccacgccttcgaccagctgcgcaacgttatcccgtccttcaacaacgacaagaagctgtccaaatatgagaccctacagatggcccagatctacatcaacgctctgtcggagttgctgcagactcccaatgtcggagagcaaccgccgccgcccacagcttcctgcaaaaatgaccaccatcaccttcgcaccgcctcctcctatgaaggaggtgcgggcgcctctgcggtagctggggctcagccagccccgggagggggcccgagacctaccccgcccgggccttgccggactcgcttctcaggcccagcttcctctgggggttactcggtgcagctggacgctttgcacttcccagccttcgaggacagggccctaacagcgatgatggcacagaaggacctgtcgccttcgctgcccgggggcatcctgcagcctgtacaggaggacaacagcaaaacatctcccagatcccacagaagtgacggagagttttccccccactctcattacagtgactctgatgaggccagttag